(19) United States
(12) Patent Application Publication
(10) Pub. No.: US 2016/0213872 A1
(43) Pub. Date: Jul. 28, 2016

(54) Comfort Enhancing Positive Airway Pressure Mask Cover

(71) Applicant: CPAP COMFORT COVER, LLC, Camas, WA (US)

(72) Inventor: John Norman Paulk, Camas, WA (US)

(73) Assignee: CPAP COMFORT COVER, LLC, CAMAS, WA (US)

(21) Appl. No.: 14/622,662

(22) Filed: Feb. 13, 2015

Related U.S. Application Data
(60) Provisional application No. 62/107,672, filed on Jan. 26, 2015.

(51) Int. Cl. A61M 16/06; A61M 16/00; A61M 16/08

(52) U.S. Cl. CPC A61M 16/0605; A61M 16/0875; A61M 16/0057; A61M 16/0816; A61M 2205/0238; A61M 2205/0216; A61M 2207/00; A61M 2209/00

(57) ABSTRACT

A method of creating a CPAP mask assembly, that makes use of a CPAP mask, including a rigid portion adapted to connect to an air-delivery hose and to provide structure and a pliable, soft, face-contacting portion, supported by the rigid portion. Also utilized is a mask cover, having an annular piece of bio-compatible, absorbent material, having an outer edge and an inner edge that defines a plain opening and an elastic loop attached about the outer edge, thereby causing the outer edge to retract inwardly. The elastic loop is then placed over the mask so that the material covers the face-contacting portion and the opening is positioned centrally. Then the elastic loop is released, permitting it to retract inwardly so that part of the rigid portion is interposed between the elastic loop and the inner edge, thereby retaining the mask cover about the rigid portion of the mask, without deforming the face-contacting portion.

### RELATED APPLICATIONS

This application claims priority from provisional application Ser. No. 62/107,672, filed Jan. 26, 2015, which is incorporated by reference as if fully set forth herein.

### BACKGROUND

Sleep apnea is a condition wherein the airway leading to the lungs closes during sleep. To treat this condition a continuous positive airway pressure machine (CPAP) may be provided. For the purposes of this application the term CPAP includes Bi-Level PAP machine (BiPAP), variable positive airway pressure (VPAP) and automatic positive airway pressure (APAP). A CPAP machine blows air through an air-delivery hose and into the nose, and thereby inflates the airway, permitting breathing to continue. Referring to FIG. 1, typically, the hose is placed into fluid connection with the nose, by way of a mask, such as mask 10, which includes a rigid portion 12, which supports a pliable, soft, face-contacting portion 14, adapted to seal an area about the nose or about both the nose and the mouth. A central opening 16 permits attachment of a rigid swivel tube, which connects to the air-delivery hose. In some masks a structure is provided to gently contact the forehead. Attachment structures 18 are provided to attach further elements that receive straps for harnessing the mask 10 to the face of a user. The face-contacting portion 14 is typically made of silicone. Unfortunately, with use the mask can become uncomfortable, particularly if it has been covered by skin oil or moisture and has shifted about on the face as a result. Even when this has not happened, the persistent pressure of the silicone against the skin can irritate the skin.

Other face masks for medical purposes, such as administering oxygen, exist or have been used in the past. Various mechanisms have been used to make this type of mask more comfortable. One of these is shown in U.S. Pat. No. 6,698,427, issued to Clowers. The partial cover that is disclosed in this patent is retained on a mask by two elastic loops. This configuration relies on the rigidity of the mask it is applied to, and if applied to a mask having a pliable, soft, face-contacting portion, would tend to deform this portion, by the elastic loop that would be interposed between the mask and the face, in a manner that would likely permit air to escape and greatly decrease patient comfort and the effectiveness of the therapy.

### SUMMARY

In a first separate aspect, a method of creating a CPAP mask assembly that facilitates comfort during CPAP treatment, that makes use of a CPAP mask, including a rigid portion adapted to connect to an air-delivery hose and to provide structure and a pliable, soft, face-contacting portion, supported by the rigid portion. Also, used is a mask cover, having an annular piece of bio-compatible, absorbent material, having an outer edge and an inner edge that defines a plain opening and an elastic loop attached about the outer edge, thereby causing the outer edge to retract inwardly. In the next step, the elastic loop is stretched and placed over the mask so that the material covers the face-contacting portion and the opening is positioned substantially centrally, relative to the face-contacting portion. Then the elastic loop is released, permitting it to retract inwardly so that part of the rigid portion is interposed between the elastic loop and the inner edge, thereby retaining the mask cover about the rigid portion of the mask, without deforming the pliable, soft face-contacting portion.

In a second separate aspect, a CPAP mask cover, comprising an annular piece of bio-compatible, absorbent material having a major axis of between 6 cm and 25 cm, and having an outer edge and an inner edge that defines a plain opening that has a major axis of between 0.6 cm and 8 cm. Also, an elastic loop is sewn into a casing formed at the outer edge, thereby causing the outer edge to retract inwardly.

In a third separate aspect, a CPAP mask assembly that facilitates comfort during CPAP treatment, comprising a CPAP mask, that includes a rigid portion adapted to connect to an air-delivery hose and to provide structure and a pliable, soft, face-contacting portion, supported by the rigid portion. A mask cover, having an annular piece of bio-compatible, absorbent material, having an outer edge and an inner edge that defines a plain opening. An elastic loop is attached about the outer edge, thereby causing the outer edge to retract inwardly, so that the mask cover is placed over the mask so that the material covers the face-contacting portion and the opening is positioned substantially centrally, relative to the face-contacting portion and the elastic loop is retracted inwardly so that part of the rigid portion is interposed between the elastic loop and the inner edge, thereby retaining the mask cover about the rigid portion of the mask, without deforming the pliable, soft face-contacting portion.

### BRIEF DESCRIPTION OF THE DRAWINGS

FIG. 1 is a top-side angle photograph of a CPAP mask.

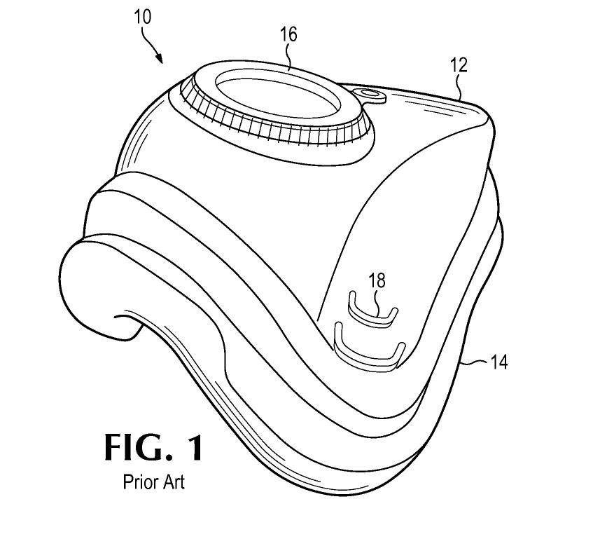

FIG. 2 is a view of a CPAP mask cover, according to the present invention.

FIG. 3 is a front-view of a CPAP mask covered by a cover, according to the present invention.

FIG. 4 is a rear-view of the CPAP mask and cover of FIG. 3, vertically rotated by 180 degrees.

FIG. 5 is a front view of a patient wearing a CPAP mask, including a cover, according to the present invention.

Exemplary embodiments are illustrated in referenced drawings. It is intended that the embodiments and figures disclosed herein are to be considered illustrative rather than restrictive.

### DETAILED DESCRIPTION OF THE PREFERRED EMBODIMENTS

Definition:

A “plain opening,” in the context of this application, is an opening which is not constricted or urged to a particular shape or size, by an elastic element, such as an elastic loop.

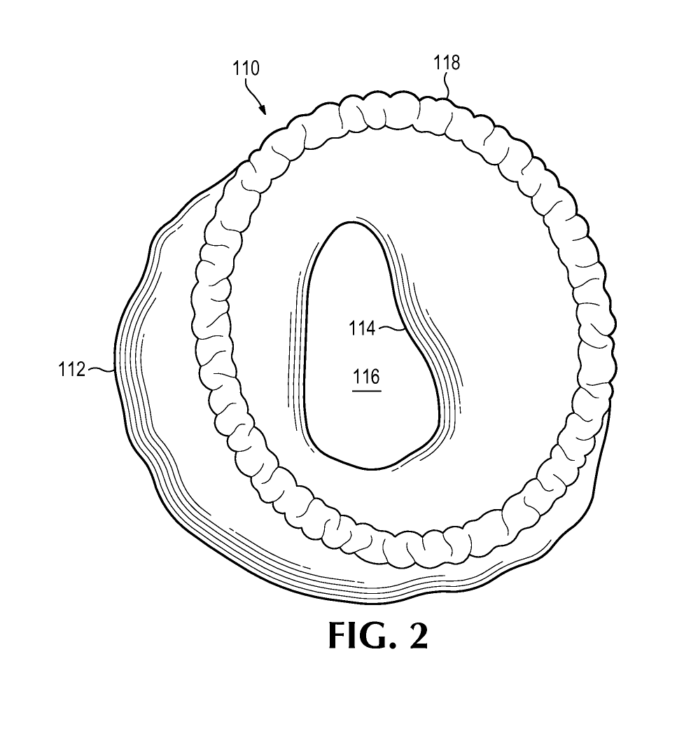

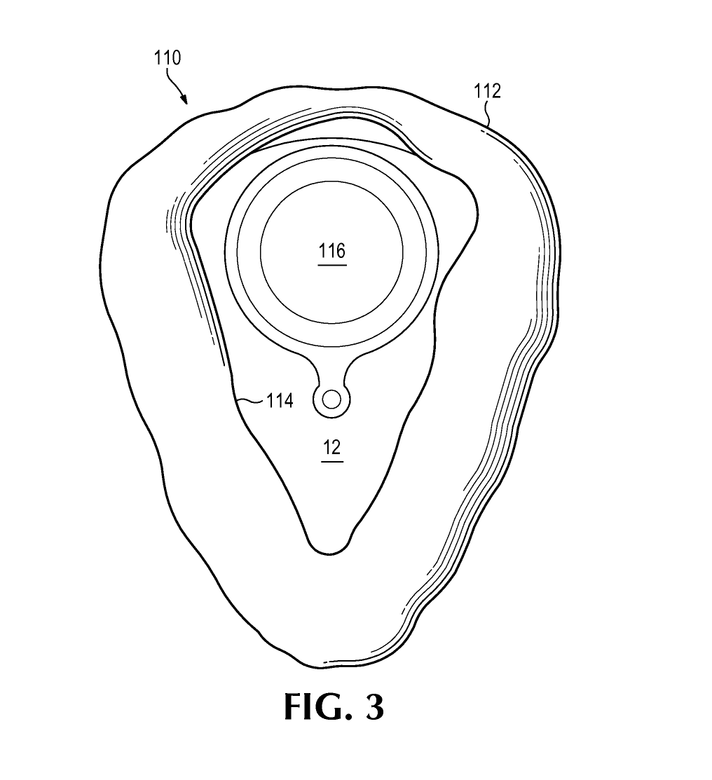

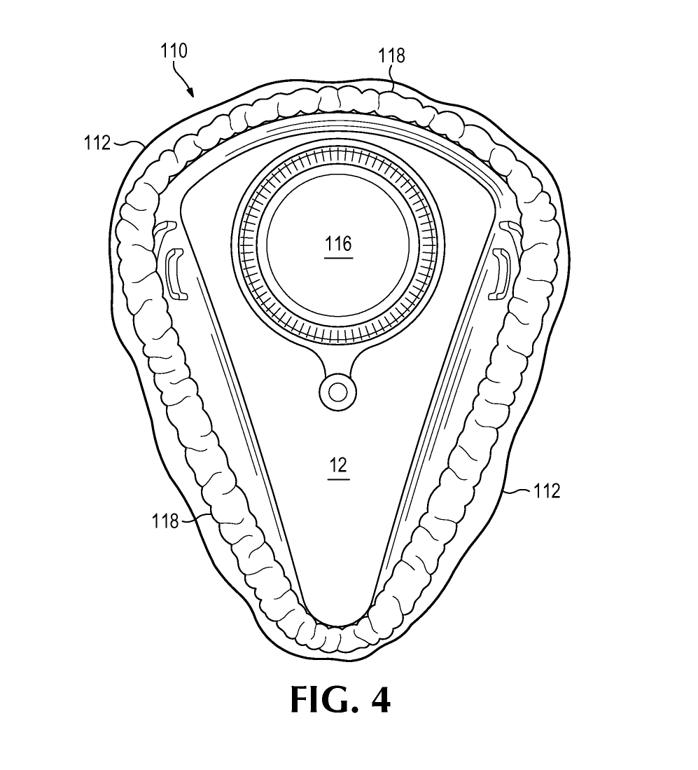

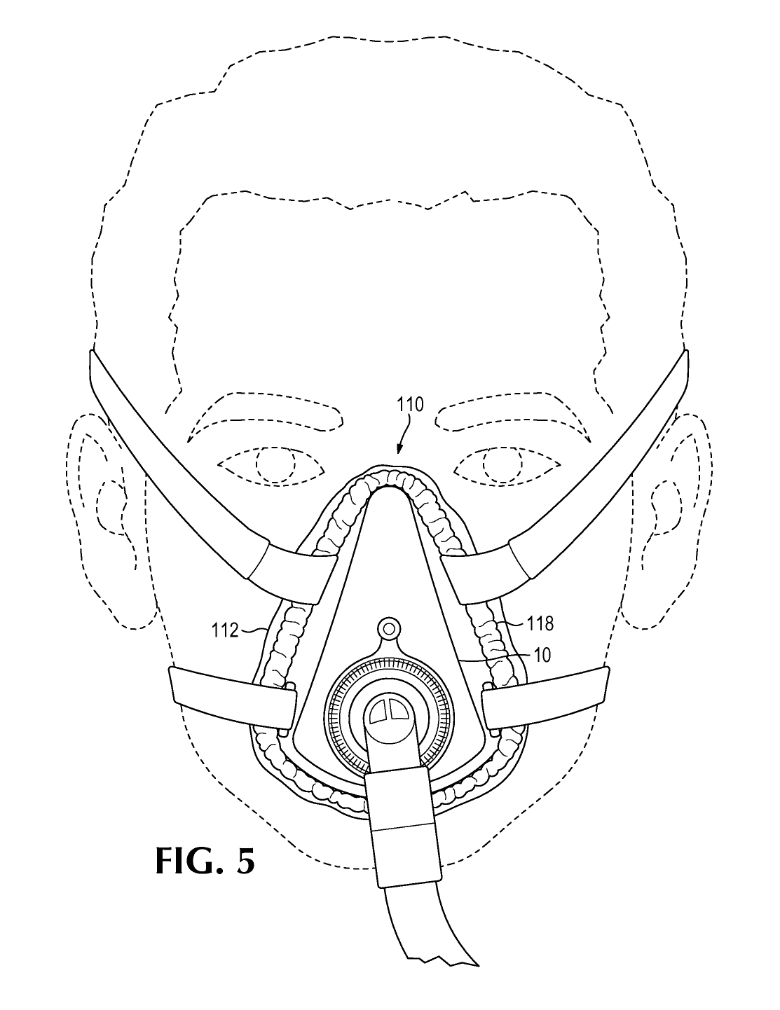

Referring to FIG. 2 a mask cover 110, includes an annular piece of material 112, having an inner edge 114, defining a plain opening 116, and an outer edge 118, and an elastic loop (not shown) sewn about said outer edge, which is hemmed and gathered about the elastic loop. This gives mask cover 110 a three dimensional, cup-like contour. Referring to FIGS. 3, 4 and 5 in use, the outer edge 118 is stretched over rigid portion 12, and is permitted to contract, so that cover 110 is retained by the elastic in outer edge 118, which is retracted so that the outer part of rigid portion 12 is interposed between outer edge 118 and inner edge 114. Plain opening 116 does not exert any force, which would act to deform soft, face-contacting portion 14. Material 112 covers the soft portion 14 of the mask 10.

The user may now sleep with the advantage of material 112 absorbing skin oil or moisture and providing a softer and more comfortable surface than portion 14 of mask 10. In one preferred embodiment, material 112 is felt textile, but in another preferred embodiment it is a woven or a knit material. Cover 110 may be made inexpensively enough so that a cover 110 could be used for a few nights and then disposed and replaced with another. It is an advantage of cover 110, however, that it can be laundered, providing an even less expensive alternative than disposing of used covers 110. In an alternative preferred embodiment an absorbent paper material, such as that used in facial tissues, may be used for material 112. In a further embodiment, a light adhesive is applied to one side of material 112, so that it adheres very gently to portion 14, to help maintain mask 10 in a preferred position, in which it has been placed.

While a number of exemplary aspects and embodiments have been discussed above, those possessed of skill in the art will recognize certain modifications, permutations, additions and sub-combinations thereof. It is therefore intended that the following appended claims and claims hereafter introduced are interpreted to include all such modifications, permutations, additions and sub-combinations as are within their true spirit and scope.

1. A method of creating a CPAP mask assembly that facilitates comfort during CPAP treatment, comprising:
a. providing a CPAP mask, including:
i. a rigid portion adapted to connect to an air-delivery hose and to provide structure; and
ii. a pliable, soft, face-contacting portion, supported by said rigid portion;
b. providing a mask cover, having:
i. an annular piece of bio-compatible, absorbent material, having an outer edge and an inner edge that defines a plain opening; and
ii. an elastic loop attached about said outer edge, thereby causing said outer edge to retract inwardly;
c. stretching said elastic loop and placing said mask cover over said mask so that said material covers said face-contacting portion and said opening is positioned substantially centrally, relative to said face-contacting portion; and
d. releasing said elastic loop, permitting it to retract inwardly so that part of said rigid portion is interposed between said elastic loop and said inner edge, thereby retaining said mask cover about said rigid portion of said mask, without deforming the pliable, soft face-contacting portion.

2. The method of claim 1, wherein said material is a knit material.

3. The method of claim 1, wherein said material is a woven material.

4. The method of claim 1, wherein said material is comprised of cotton.

5. The method of claim 1, wherein said elastic loop is contained in a casing in said material, which is gathered about said elastic loop.

6. The method of claim 1 further being a method of ongoing maintenance of said mask assembly, further including after use of said mask assembly, removing said mask cover from said mask and laundering said mask cover.

7. The method of claim 6, further including using said mask cover, in a process of steps a, b, c, and d of claim 1, again after laundering.

8. A CPAP mask cover, comprising:
a. an annular piece of bio-compatible, absorbent material having a major axis of between 6 cm and 25 cm, and having an outer edge and an inner edge that defines a plain opening that has a major axis of between 0.6 cm and 8 cm; and
b. an elastic loop sewn into a casing formed at said outer edge, thereby causing said outer edge to retract inwardly.

9. The method of claim 8, wherein said material is a knit material.

10. The method of claim 8, wherein said material is a woven material.

11. The method of claim 8, wherein said material is comprised of cotton.

12. The method of claim 8, wherein said elastic loop is contained in a casing in said material, which is gathered about said elastic loop.

13. A CPAP mask assembly that facilitates comfort during CPAP treatment, comprising:
a. a CPAP mask, including:
i. a rigid portion adapted to connect to an air-delivery hose and to provide structure; and
ii. a pliable, soft, face-contacting portion, supported by said rigid portion;
b. a mask cover, having:
i. an annular piece of bio-compatible, absorbent material, having an outer edge and an inner edge that defines a plain opening; and
ii. an elastic loop attached about said outer edge, thereby causing said outer edge to retract inwardly; and
c. wherein said mask cover is placed over said mask so that said material covers said face-contacting portion and said opening is positioned substantially centrally, relative to said face-contacting portion and said elastic loop is retracted inwardly so that part of said rigid portion is interposed between said elastic loop and said inner edge, thereby retaining said mask cover about said rigid portion of said mask, without deforming the pliable, soft face-contacting portion.

14. The assembly of claim 13, wherein said material is a knit material.

15. The assembly of claim 13, wherein said material is a woven material.

16. The assembly of claim 13, wherein said material is comprised of cotton.

17. The assembly of claim 13, wherein said elastic loop is contained in a casing in said material, which is gathered about said elastic loop.
